(19) United States
(12) Patent Application Publication
(10) Pub. No.: US 2006/0280761 A1
(43) Pub. Date: Dec. 14, 2006

(54) Nanofluidized B-12 composition and process for treating pernicious anemia

(75) Inventors: Russell Van de Casteele, Deerfield Beach, FL (US); Michael Gerike, Pompano Beach, FL (US)

Correspondence Address: MCHALE & SLAVIN, P.A., 2855 PGA BLVD, PALM BEACH GARDENS, FL, 33410, US

(73) Assignee: Health Plus International, Inc.

(21) Appl. No.: 11/369,706

(22) Filed: Mar. 7, 2006

Related U.S. Application Data
(63) Continuation-in-part of application No. 11/056,027, filed on Feb. 11, 2005, now abandoned.
Application No. 11/056,027 is a division of application No. 10/096,337, filed on Mar. 11, 2002, now Pat. No. 6,861,066.

(51) Int. Cl. A61K 9/00; A61K 31/714

(52) U.S. Cl. 424/400; 514/52; 977/906

(57) ABSTRACT

A method of manufacturing a stable nanosuspension for delivery of a biologically active agent, particularly vitamin B-12, into the bloodstream of a subject is disclosed. A nanofluidizable mixture containing vitamin B-12 is initially formed and processed via a nanofluidization process to form the stable nanosuspension, which may be administered via the transmucosal membranes or other suitable routes of administration. This product demonstrates increased bioavailability, enhanced period of onset, and enhanced stability for a controlled-release product.

CROSS REFERENCE TO RELATED APPLICATION

This application is a continuation-in-part of co-pending application Ser. No. 11/056,027, filed Feb. 11, 2005, which is a division of application Ser. No. 10/096,337, filed Mar. 11, 2002, now issued U.S. Pat. No. 6,861,066, the contents of which are herein incorporated by reference.

### FIELD OF THE INVENTION

The present invention relates to the administration of biologically active agents and more particularly to a method for enhancing absorption of an agent into the bloodstream by forming, via a nanofluidization technique, a highly solulable and stable uniform submicron emulsion or nanosuspension, for delivery of biologically active agents, particularly B-12 vitamin, by multiple pathways, particularly via mucosal membranes.

### BACKGROUND OF THE INVENTION

Biologically active agents such as nutritional supplements, hormones, and a variety of pharmaceutical preparations, which will generally be referred to as “drugs” are typically provided in oral (liquids or solids) or injectable dosage formulations, however there are many disadvantages associated with this type of administration.

Many of the ingredients are degraded within the gastrointestinal (GI) tract or undergo first-pass metabolism in the liver. In addition, there exists a segment over 20% of the population (e.g., children, elderly, infirm, etc.) who experience difficulty swallowing pills or are unable to tolerate any solids.

During the past three decades formulations that control the rate and period of drug delivery (e.g., time-release medications) and target specific areas of the body for treatment have become increasingly common and complex. Some have provided solutions to the problem of administering different types of drugs but there are still a large number of medications that do not achieve maximum pharmaceutical effect because they do not reach the intended tissue targets either fast enough or in high enough concentrations.

The potency and therapeutic effects of many drugs are limited or reduced because of the partial degradation that occurs before they reach a desired target in the body. Further, injectable medications could be made less expensively and administered more easily if they could simply be dosed by other routes such as the mucosal membranes, including but limited to, the oral mucosa, the pulmonary mucosa or through the vaginal and intestinal mucosa. However, this improvement cannot happen until methods are developed to safely shepherd drugs through these specific areas of the body, where different physiological environments (e.g. low pH values in the stomach) can destroy a medication or where absorption is not rapid or complete, or through an area where healthy tissue might be adversely affected.

Transmucosal routes of drug delivery offer distinct advantages. Of the various routes, the mucosal linings of the nasal passages and the oral cavity are the most attractive. Although the nasal route has reached commercial success with several drugs, such as with allergy medications, potentially serious side-effects, such as irritation and possibly irreversible damage to the ciliary action of the nasal cavity from chronic application, have deterred health professionals from recommending their long-term use.

Within the oral cavity there are three generally recognized routes of administration of a biologically active agent. The first route is local delivery, mainly limited to applications regarding disruptions occurring within the oral cavity itself, such as a canker sore. The second route is sublingual delivery, wherein transfer of a biologically active agent is achieved through the mucosal membranes lining the floor of the mouth, which provides rapid absorption and has reached commercial status with biologically active agents such as nitroglycerin, which is placed under the tongue. Because of the high permeability and the rich vascular supply, transport via the sublingual route results in a rapid onset of action, providing a delivery route appropriate for highly permeable drugs with short delivery period requirements and an infrequent dosing regimen. Unfortunately, it produces a saliva wash (swallowing) and in the case of nitroglycerin it has been found to cause headaches as a result of administering amounts of the drug in excess of that needed for the desired pharmacological effect.

The third generally recognized route is the buccal mucosa. This area encompasses the mucosal membranes of the inner lining of the cheeks. This area also has a rich blood supply, is robust, and provides a short cellular recovery time following stress or damage. Although the buccal mucosa is less permeable than the sublingual area, the expanses of a smooth and relatively immobile mucosa provide a highly desirable absorption pathway for sustained-release and controlled-release delivery of biologically active agents. As with other transmucosal routes of administration, two major advantages include avoiding hepatic first-pass metabolism and pre-systemic elimination within the GI tract.

One of the disadvantages associated with buccal mucosa delivery of a biologically active agent has been the relatively low passage of active agents across the mucosal epithelium, resulting in low agent bioavailability, which translates into a substantial loss of usable active agent within each dosage. Various permeation and absorption enhancers such as polysorbate-80, sorbitol, and rphosphatidylcholine have been explored to improve buccal penetration. Studies indicate that the superficial layers and protein domain of the epithelium may be responsible for maintaining the barrier function of the buccal mucosa (Gandhi and Robinson, Int. J. Pharm. (1992) 85, pp. 129-140).

Additionally, it is known that use of a permeation enhancer can increase the passage of a biomolecule. Furthermore, studies have suggested the feasibility of buccal delivery of even a rather large molecular weight pharmaceutical (Aungst and Rogers, Int. J. Pharm. (1989) 53, pp. 227-235).

An additional area of investigation includes the use of bioadhesive polymers in buccal delivery systems. Bioadhesive polymers have been developed to adhere to a biological substrate in order to maintain continual contact of an agent with the delivery site. This process has been termed mucoadhesion when the substrate is mucosal tissue (Ch'ng et al., J. Pharm. Sci. (1985) 74, 4, pp. 399-405).

The goal of all drug delivery systems is to deploy medications intact to specifically targeted parts of the body through a medium that can control the therapy's administration by means of either a physiological or chemical trigger. To achieve this goal, a number of researchers have turned to advances in micro- and nanotechnology. One prominent area of endeavor is the production of so-called “nanoparticles” which act as chemical or physical “carriers” of drugs.

During the past decade, novel polymeric microspheres, polymer micelles, and hydrogel-type materials have been shown to be effective in enhancing drug targeting specificity, lowering systemic drug toxicity, improving treatment absorption rates, and providing protection for pharmaceuticals against biochemical degradation. These are all goals of efficient drug delivery. In addition, several other experimental drug delivery systems show signs of promise, including those composed of biodegradable polymers, dendrimers (so-called star polymers), electroactive polymers, and modified C-60 fullerenes (also known as “buckyballs”.)

Polymer drug delivery systems are based on “carriers” which are composed of mixing polymeric chemical compounds with drugs to form complex, large molecules, which “carry” the drug across physiological barriers.

Illustrative examples of these polymeric compounds include but are not limited to poly(ethylene-glycol)-poly(alpha, beta-aspartic acid), carboxylates, and heterobifunctional polyethylene glycol.

Another type of nanotechnology revolves around the use of “hydrogels” as carriers of drugs. The principle behind this technology is to use a chemical compound which traps a drug and then releases the active compound by “swelling” or expanding inside of specific tissues, thus allowing a higher concentration of the drug in a biodegradable format. Hydrogels are very specialized systems and are generally formulated to meet specific needs for the delivery of individual drugs.

During the past two decades, research into hydrogel delivery systems has focused primarily on systems containing polyacrylic acid (PAA) backbones. PAA hydrogels are known for their super-absorbency and ability to form extended polymer networks through hydrogen bonding. In addition, they are excellent bioadhesives, which means that they can adhere to mucosal linings within the gastrointestinal tract for extended periods, releasing their encapsulated medications slowly over time.

One example of the complexity of these systems is a glucose-sensitive hydrogel that could be used to deliver insulin to diabetic patients using an internal pH trigger. This system features an insulin-containing “reservoir” formed by a poly[methacrylic acid-g-poly(ethylene glycol)]hydrogel membrane into which glucose oxidase has been immobilized. The membrane itself is housed between nonswelling, porous “molecular fences”.

Although these approaches are the focus of intense research, other processes are also under consideration, including aerosol inhalation devices, transdermal methodologies, forced-pressure injectables, and biodegradable polymer networks designed specifically to transport new gene therapies.

Another method to formulate drugs for delivery has been the use of nanosuspensions of drugs to reduce size and create uniform suspensions. The use of commercial devices such as mill processors, microfluidizers and homogenizers has allowed the formulation of nanosuspensions of various substances. Nanosuspended drugs can also be wrapped in liposomes or made into micellar mixtures by mixing the drug preparations with appropriate chemical compounds.

Prior artisans have explored a variety of avenues in an effort to produce a viable and efficient means for transmucosal delivery. Such avenues include the use of liposomal carriers to enhance uptake or facilitate the delivery of a product, decreasing the particle size of microspherical carriers, or employing a physical matrix, such as a sponge, to hold a medicinal product at the buccal area.

What has been lacking in the art is a stable vitamin B-12 composition which is uniquely efficacious in the accelerated formation and maturation of red blood cells; a method, via the use of the novel composition, for treating various forms of anemia, particularly pernicious anemia; and a method for increasing the bioavailability and biological activity of vitamin B-12, most particularly when administered by a route, such as the transmucosal or oral mucosa membranes, whereby metabolic and gastrointestinal interference is avoided.

### DESCRIPTION OF THE PRIOR ART

U.S. Application Publication No. 2003/0072801 discloses a solubility-improved drug form combined with a concentration-enhancing polymer in a sufficient amount so that the combination provides substantially enhanced drug concentration in a use environment relative to a control comprising the same amount of the same drug form without the concentration-enhancing polymer. Unlike the instant process, the reference requires drug encapsulation in polymers for effective drug delivery.

U.S. Pat. No. 5,681,600 discloses a stable, liquid nutritional product and a method for its manufacture. Preparation of the product comprises forming a protein solution, a carbohydrate solution, and an oil blend to combine with an amount of a nutritional ingredient containing soy polysaccharide. Soy polysaccharide is essential as a stabilizer to maintain the components in solution, thereby avoiding the need for carrageenan, and to reduce the need to overfortify the amount of nutritional ingredient included, owing to inherent degradation over time. The combined solution is subjected to microfluidization as an alternative to homogenization. The reference fails to suggest forming a stable uniform submicron emulsion as a means for increasing bioavailability and stability of the final product.

U.S. Pat. No. 5,056,511 discloses a method for compressing, atomizing, and spraying liquid substances for inhalation purposes. The liquid substance is compressed under high pressure to reduce its volume. The released liquid is then atomized to cause the liquid substance to burst into particles in the size range of about 0.5 μm to about 10 μm, thereby forming a very fine cloud for direct inhalation by the end-user. This method is intended for immediate use, and does not provide a product having the stability of the product disclosed herein. The reference also fails to suggest forming a stable uniform submicron emulsion as a means for increasing bioavailability and stability of the final product.

U.S. Pat. No. 4,946,870 discloses a film-forming delivery system, which requires at least one aminopolysaccharide, useful for delivery of pharmaceutical or therapeutic active agents to a desired topical or mucous membrane site. The delivery of active agent may be in the form of a gel, patch, sponge, or the like.

U.S. Pat. No. 5,891,465 discloses the delivery of a biologically active agent in a liposomal formulation for administration into the mouth. The phospholipid vesicles of the liposomal composition provide an increase in bioavailability of the biologically active agent in comparison to an oral dosage form. The liposomal composition, while reaching a submicron level for absorption into the bloodstream, nevertheless requires specific components to be provided within a narrow range of concentration in order to enable the one or more bilayer forming lipids to achieve delivery through the mucosal lining.

U.S. Pat. No. 5,981,591 discloses a sprayable analgesic composition and method of use. The sprayable dosage includes one or more surfactants for facilitating the absorption through the surface of the buccal mucosa of the mouth. The use of surfactants for increasing bioavailability is of limited value, since they are only effective for a small proportion of biologically active agents. The reference fails to provide a stable uniform submicron emulsion, thus failing to achieve the enhancements in absorption time, bioavailability, stability, and controlled-release demonstrated by the instant invention.

Drug preparations called nanosuspensions were produced by high-pressure homogenization, and are the subject of U.S. Pat. No. 5,858,410 to Muller.

Prior to the use of high pressure homogenization, nanosuspensions were prepared by a pearl milling process, which was a longer process than pressure homogenization. This technology is the subject of U.S. Pat. No. 5,271,944 to Lee. A number of other methods have been used to prepare nanosuspensions with various degrees of success including low energy agitators, turbine agitators, colloid mills, sonolators, orifices, media mills, rotor stator mixers and sonicators.

There is no suggestion in the prior art regarding the production of nanosuspensions via a nanofluidization technique nor for the use of said nanosuspensions for the delivery of biologically active agents, e.g. B-12 vitamins; and conspicuously lacking is any suggestion of administration of the biologically active agent containing nanosuspensions via a transmucosal membrane or oral mucosa membrane (e.g. buccal mucosal route).

### SUMMARY OF THE INVENTION

The present invention is directed toward a method for creating nanostructured preparations of vitamin B-12, which define stable uniform submicron emulsions, or nanosuspensions, which have been demonstrated herein to enable enhanced delivery of vitamin B-12 into the bloodstream of a subject, while also accelerating the formation of viable red blood cells in patients suffering from deficiency or anemia.

The nanostructured preparations of the instant invention, which are at times referred to as nanofluidized, can be prepared as aqueous or organic solutions, or as emulsions using known emulsifying agents, and can be delivered by way of a nanofluidized spray, an aerosol, a tablet, a pill, a liquid, a suppository, or a gel. Preferably, delivery is transmucosal, however, delivery may be accomplished by parenteral, intrathecal, intravenous, transdermal, and any or all commonly recognized methods for drug delivery.

The instant inventor has utilized a nanofluidization technique for the production of nanosuspensions of aqueous and oil-based solutions for use in drug delivery systems. The instant process does not require encapsulation in polymers or the use of hydrogels or other supporting or encapsulating substances. Chemicals prepared in this manner are called “nanosuspensions.” This process allows molecules to be embedded into micro- or nanodroplets of between about 10 μm to about 87 nm in size, which are used to create stable and uniform emulsions and dispersions.

The nanosuspensions of the instant invention are effective in providing higher concentrations of a molecule in the bloodstream over a longer period of time as compared to molecules prepared by other pharmacological methods and similarly delivered (e.g., by a oral mucosal route, intestinal absorption, or the like). While not wishing to be bound to any particular theory of operation, it has been hypothesized that the nanosuspensions of the instant invention allow molecules to be delivered across tissue barriers at a more even rate than non-nanofluidized preparations.

In its broadest context, the method includes mixing together various aqueous and/or non-aqueous components (e.g., organic or inorganic components) and forming at least one solution. Depending upon the solubility of the biologically active agent, a nanofluidizable mixture may be obtained by adding the agent to an aqueous solution, an organic solution, or a crude emulsion, which is a mixture of said organic and inorganic solutions. The nanofluidizable mixture may further contain various components such as flavorings, preservatives, surfactants, and permeation enhancers known in the art. Nanofluidizing said mixture provides a means for the mixture to form a stable uniform submicron emulsion. It is this emulsion which provides for the enhanced period of onset, bioavailability, and controlled-release capability of the final product. Upon contact of the instant emulsion with the body (e.g. with an area of the oral cavity including the buccal mucosa and sublingual membranes), the agent is absorbed into the bloodstream in an amount sufficient to elicit a desired biological response.

Accordingly, it is an objective of the instant invention to provide a new and highly efficacious form of vitamin B-12, in the form of a stable uniform nanosuspension, produced via a nanofluidization process, and effective for administration via various routes, particularly transmucosal membranes.

It is a further objective of the instant invention to provide a preparation, containing vitamin B-12 as a biologically active agent, in the form of a stable uniform nanosuspension, produced via a nanofluidization process, and effective for ameliorating pernicious anemia by accelerated normalization of red blood cell physiology.

It is yet another objective of the instant invention to provide a biologically active agent containing vitamin B-12 capable of submicron stability.

It is a still further objective of the invention to provide a biologically active agent containing vitamin B-12 capable of providing increased bioavailability through various transmucosal routes, particularly the oral mucosal route.

It is a further objective of the instant invention to provide a biologically active agent containing vitamin B-12 capable of sustained-release or controlled-release action.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objectives and features thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A is a graphic comparison of mucosal absorption of nanofluidized B-12 versus gastro-intestinal absorption via oral administration of a well recognized commercially available B-12 in tablet form, and is indicative of the relative increase in circulating B-12 concentration in picograms/mL as a function of time;

FIG. 1B is a graphic comparison of mucosal absorption of nanofluidized B-12 versus gastro-intestinal absorption via oral administration of a well recognized commercially available B-12 in tablet form, and is indicative of the percent change in circulating B-12 concentration as a function of time;

FIG. 2 is a summary of initial hematological results obtained from a whole blood sample from a 65-year-old male patient, suffering from pernicious anemia induced by vitamin B-12 deficiency, prior to the sublingual administration of nanoprocessed SPRAY FOR LIFE vitamin B-12 Energy Booster of the present invention;

FIG. 3 is a photograph of the sample analyzed in FIG. 2 on a test slide under a high-powered. microscope, illustrating erythrocyte abnormalities (anisocytosis and ovalocytes);

FIG. 4 is a summary of initial hematological results obtained from a whole blood sample from a 37-year-old female patient, suffering from pernicious anemia induced by vitamin B-12 deficiency, prior to the sublingual administration of nanoprocessed SPRAY FOR LIFE vitamin B-12 Energy Booster of the present invention;

FIG. 5 is a photograph of the sample analyzed in FIG. 4 on a test slide under a high-powered microscope, illustrating an erythrocyte abnormality (macrocytosis);

FIG. 6 is a summary of initial hematological results obtained from a whole blood sample from a 57-year-old male patient, suffering from vitamin B-12 deficiency, prior to the sublingual administration of nanoprocessed SPRAY FOR LIFE vitamin B-12 Energy Booster of the present invention;

FIG. 7 is a summary of final hematological results obtained from a whole blood sample from the 65-year-old male patient in FIGS. 2 and 3, subsequent to the sublingual administration of nanoprocessed SPRAY FOR LIFE vitamin B-12 Energy Booster of the present invention for a 30-day test period;

FIG. 8 is a photograph of the sample analyzed in FIG. 7 on a test slide under a high-powered microscope, illustrating normal erythrocyte size and shape;

FIG. 9 is a summary of final hematological results obtained from a sample from the 37-year-old female patient in FIGS. 4 and 5, subsequent to the sublingual administration of nanoprocessed SPRAY FOR LIFE vitamin B-12 Energy Booster of the present invention for a 30-day test period;

FIG. 10 is a photograph of the microscope slide of the sample analyzed in FIG. 9 on a test slide under a high-powered microscope, illustrating normal erythrocyte size and shape; and

FIG. 11 is a summary of final hematological results obtained from a sample from the 57-year-old male patient in FIG. 6, subsequent to the sublingual administration of nanoprocessed SPRAY FOR LIFE vitamin B-12 Energy Booster of the present invention for a 30-day test period.

### DETAILED DESCRIPTION OF THE INVENTION

Detailed embodiments of the instant invention are disclosed herein, however, it is to be understood that the disclosed embodiments are merely exemplary of the invention, which may be embodied in various forms. Therefore, specific functional and structural details disclosed herein are not to be interpreted as limiting, but merely as a basis for the claims and as a representative basis for teaching one skilled in the art to various employ the present invention in virtually any appropriately detailed structure.

This application is directed towards a novel application of nanosuspensions for delivery of biological agents, either singly or in various combinations, e.g. multi-vitamin/mineral supplements. As an illustrative, albeit non-limiting example, the inventors have demonstrated that a common vitamin, B-12, when administered as a spray achieves higher concentrations in the blood prepared as a nanosuspension when compared to, the same non-processed vitamin B-12 administered in tablet form and absorbed gastro-intestinally. By extension, this application applies to all biologically active agents.

The terms “biologically active agent”, “biological agent”, or “agent”, are used interchangeably herein and refer to any synthetic or natural element or compound, protein, cell, or tissue including a pharmaceutical, drug, therapeutic, nutritional supplement, herb, hormone, or the like, or any combinations thereof, which when introduced into the body causes a desired biological response, such as altering body function or altering cosmetic appearance.

The terms “vitamin B-12” or “B-12” are used interchangeably herein and refer to any supplemental form known to the skilled artisan including, albeit not limited to: cyanocobalamin, methylcobalamin, adenosylcobalamin, conjugates, mixtures or combinations thereof.

While not wishing to be bound to any particular theory of operation, there are several hypothetical mechanisms that may account for the increased absorption of B-12, or alternative biologically active agents, when formulated as a nanosuspension and administered via the transmucosal route.

1. There is a greater concentration of drug at the active mucosal surface with two possible explanations for this phenomenon):

a. Increased Saturation at the Mucosal Membrane

The reduced size of the nanodroplets in the nanosuspension (which concentrates more molecules in a smaller unit volume of fluid) allows a greater number of molecules to come into contact with the mucosal membrane, over a shorter period of time. This increases the adhesiveness of the drug to the surface of the membrane and enhances the probability that more molecules will be absorbed than from non-nanofluidized preparations;

b. Increased Passive Infusion

As a result of the increased local concentration of the drug, there may be greater passive diffusion gradient across the mucosal membrane, ultimately resulting in greater levels in the plamsa.

2. Nanosuspensions stimulate active transport of the molecules across the mucosal membrane:

In adopting this explanation, it is theorized that the nanodroplets could stimulate greater “active transport” of compounds across the mucosal membrane by bringing a greater concentration of B-12 into contact with specific receptor sites.

The present invention provides a method for the delivery of a biologically active agent enhanced by the formation of a stable uniform submicron emulsion, termed a nanosuspension. While illustrative examples are limited to human subjects, the technology is in no way limited by said examples. The nanosuspensions which are the subject of the instant invention are contemplated for use in either a medical or veterinary setting, and may be administered in any reasonable fashion as is known in the art. The preferred embodiment, as thoroughly illustrated Example 1 below, is preferably formulated such that it may be sprayed into the mouth of a human subject or an animal, whereby absorption via the oral mucosa is accomplished.

In the present invention it is preferred to convert the mixture to the stable uniform submicron emulsion through the process of nanofluidization, wherein the mixture is subjected to an ultra-high energy-mixing device. One such mixing device is MICROFLUIDIZER (Microfluidics Corporation, Newton, Mass.), which provides high shear rates, maximizing the energy-per-unit fluid volume to produce uniform submicron particle and droplet sizes of chemical or particulate substances.

Process pressures are highly variable, ranging from a low of 1,500 to 40,000 psi, enabling the processing of a wide variety of fluids ranging from simple oil-in-water emulsions to high-weight-percent solids-in-liquid suspensions.

The MICROFLUIDIZER contains an air-powered intensifier pump designed to supply the desired pressure at a constant rate to the product stream. As the pump travels through its pressure stroke, it drives the product at a constant pressure through precisely defined fixed-geometry microchannels within the interaction chamber. As a result, the product stream accelerates to high velocities, creating shear rates within the product stream that are orders of magnitude greater than any other conventional means. All of the product experiences identical processing conditions, producing the desired results, including uniform particle and droplet size reduction, often submicron.

As a result of the high shear rate there is produced a mixture containing uniform submicron particles and the creation of stable emulsions and dispersions is achieved. This processing overcomes limitations of conventional processing technologies by utilizing high pressure streams that collide at ultra-high velocities in precisely defined microchannels. The final product is a stable uniform submicron emulsion, a “nanosuspension” composed of nanodroplets.

The stability and rate of absorption may be further enhanced by one or more components within the initial emulsion. In addition, the rate of absorption of the final product may be enhanced by the uniformity or size of the particles.

Permeation enhancers utilized in the present invention include the conventional physiologically acceptable compounds generally recognized as safe (GRAS) for human consumption. Any surfactant which assists in decreasing particle size is contemplated by the instant invention.

In order to examine the increased efficiency of absorption this formulation provides, an initial experimentation was performed (Example 1). An additional experiment was performed to demonstrate the efficacy of the formulation in patients with pernicious anemia (Example 2) below.

Vitamin B-12 is a water-soluble, B-complex vitamin that facilitates DNA and RNA synthesis, amino acid and protein metabolism, nerve cell and red blood cell development and function, (e.g., hemoglobin synthesis and oxygen transport). Vitamin B-12 is composed of a corrin ring structure that surrounds an atom of cobalt; hence, B-12 is also known as cobalamin.

The richest dietary source of vitamin B-12 is animal liver. Eggs, cheese and some species of fish also supply a small amount; vegetables and fruits are very poor sources of vitamin B-12. Most deficiencies of vitamin B-12 result from an impaired ability of the gastrointestinal tract to produce a transport protein called the “intrinsic factor”, which is needed to absorb the vitamin from the small intestine. Such inabilities to absorb B-12 frequently occur with the onset of advanced age, pernicious anemia, gastric conditions, or surgery. When therapeutically relevant doses of B-12 are not achieved, supplementation is often required by way of injection or orally. Often, oral supplementation with vitamin B-12 is preferred as it is safe, efficient, inexpensive and less painful than injection.

Characteristic symptoms of B-12 deficiency cause wide-ranging and serious symptoms that include fatigue, weakness, nausea, constipation, flatulence, weight loss, insomnia, and loss of appetite. Deficiency can also lead to neurological problems such as numbness, cramping and tingling in the extremities. Additional symptoms of B-12 deficiency include difficulty in maintaining equilibrium, depression, confusion, poor memory, and soreness of the mouth or tongue.

A nanofluidizable mixture with vitamin B-12 as the biologically active agent was prepared according to the following procedure:

an aqueous solution was formed from about 83.0% (wt/wt) of purified water in an appropriately sized mixing vessel. To this mixing vessel approximately 0.13% (wt/wt) vitamin B-12 (cyanocobalamin) was added and stirred for about 10 minutes.

Next, about 10.0% wt/wt vegetable glycerin (acting as a solvent and taste enhancer) was stirred into the aqueous solution. Spearmint flavor (taste enhancer) at about 1.0% wt/wt, citric acid (as an acidulent/buffering agent) at about 1.0% wt/wt, polysorbate-80 (an emulsifier and surface activator) at about 2.0% wt/wt was added, potassium hydroxide (pH balancer) at about 3.0% wt/wt, and potassium sorbate (a preservative) at about 0.20% wt/wt were also added to the mixing vessel. Upon reaching complete dissolution, the compound emulsion appeared homogeneous, red-purple, and slightly transparent with a measured pH of about 4.0 to about 5.0 and specific gravity (g/ml) of about 1.08 to about 1.15.

The crude emulsion was then processed through a model M-110Y MICROFLUIDIZER (Microfluidics Corporation, Newton, Mass.) under 21 kpsi. After a single pass, the mean particle size, according to a Horiba LA-910 particle size analyzer, was 188 nm. The appearance of the solution did not change after processing.

The resulting stable uniform submicron emulsion was then placed into a spray vial with a fine mist nozzle. The particular nozzle provided thorough coverage of the oral cavity.

### Example 1

Absorption of Nanoprocessed vitamin B-12 versus Oral administration of commercial vitamin B-12 in tablet form in a normal human subject.

Objective:

To evaluate the absorption rate of nanoprocessed vitamin B-12 across the buccal mucosa when administered by a spray applicator, compared to a well recognized commercially available vitamin B-12 in tablet form, in a normal healthy subject. It should be noted that attempts were made to additionally compare the absorption rate of the nanoprocessed vitamin B-12 across the buccal mucosa when administered by a spray applicator, as set forth in the instant invention to a tableted vitamin B-12 constructed and arranged for sublingual dissolution and subsequent absorption. Unfortunately, such a comparison could not be practically performed owing to the fact that the equivalent dosing could not be administered via sublingual tablet in a quick and efficient manner without incurring a high degree of swallowing of the product which concomitantly led to unwanted absorption via the gastrointestinal tract.

Utilizing a process, as outlined above, for producing nanodroplets of aqueous and oil based solutions for use in drug delivery systems, nanosuspension formulations for testing were produced. The process allows vitamin B-12 molecules to be embedded into micro- or nanodroplets of between about 10 μm and about 188 nm in size, which are used to create stable and uniform emulsions and dispersions.

Theoretically, such dispersions should allow molecules to be delivered across tissue barriers at a more even rate than non-nanofluidized or “normal” solutions. This should allow the accumulation of higher concentrations of a molecule in the bloodstream over a longer period of time than with molecules prepared by standard pharmacological methods and delivered either by transmucosal or intestinal absorption.

By using the “nanofluidization” process to prepare mixtures of biologically active agents, (e.g. vitamins, minerals, and other nutritional supplements) may be designed, manufactured and standardized for use in spray applicators which deliver single dose sprays which may be absorbed transmucosally. The purpose of this type of delivery is to introduce such biologically active agents into the body in a manner which allows, over time, more rapid, uniform, and complete absorption than that which has been heretofore achieved via administration of non-nanofluidized components in the form of sprays, aerosols, pills, tablets, capsules, suppositories, gels, or liquids which are absorbed through the gastrointestinal tract. Apart from the absorption efficiencies, the nanofluidization process appears to offer increases in shelf life, with testing showing a shelf life of about 3 years.

Methods:

The normal human subject used in this study had not taken any supplements containing B-12 for one month prior to testing or between visits, and avoided all dairy and meat products. Approximately 5 ml of blood was drawn by routine venipuncture to establish a baseline (pre-dosing). A spray applicator was used to administer a single megadose of 15 mgs (15,000 mcg) of nanoprocessed SPRAY FOR LIFE vitamin B-12 Energy Booster of the present invention by carefully spraying the inside of each individual's two cheeks (buccal mucosa) five times. Next, serial blood draws were obtained at 7.5, 15, 30, 60, 120, 180, 240, 300, 360 and 480 minutes after administration.

Preparations of nanoprocessed SPRAY FOR LIFE vitamin B-12 Energy Booster of the instant invention and a well recognized commercially available non-processed vitamin B-12 in tablet form were administered at different times to the same individual, subsequent to a period of time to enable washout (e.g. 1 week), thereby allowing an intrasubject comparison.

30 well recognized commercially available tablets equivalent to approximately 15 mgs (15,000 mcg) of vitamin B-12 were swallowed by the test subject, as instructed, and allowed to absorb via the gastro-intestinal tract. Again, serial blood draws were obtained at 7.5, 15, 30, 60, 120, 180, 240, 300, 360 and 480 minutes after administration.

Vitamin B-12 was assayed from the whole blood samples in a commercial laboratory using an Access Immunoassay system (Beckman Coulter, Inc., Fullerton, Calif.).

Results:

Data was recorded showing both the rate and amount of vitamin B-12 adsorption in the test subject after administration of nanoprocessed SPRAY FOR LIFE vitamin B-12 Energy Booster of the instant invention via the buccal mucosa and in vitamin B-12 tablets via the gastro-intestinal tract. Normal blood levels of vitamin B-12 in individuals who have not taken supplements or who have not recently eaten foods high in vitamin B-12 concentration within 24-48 hours are between about 200 and 900 picograms/ml.

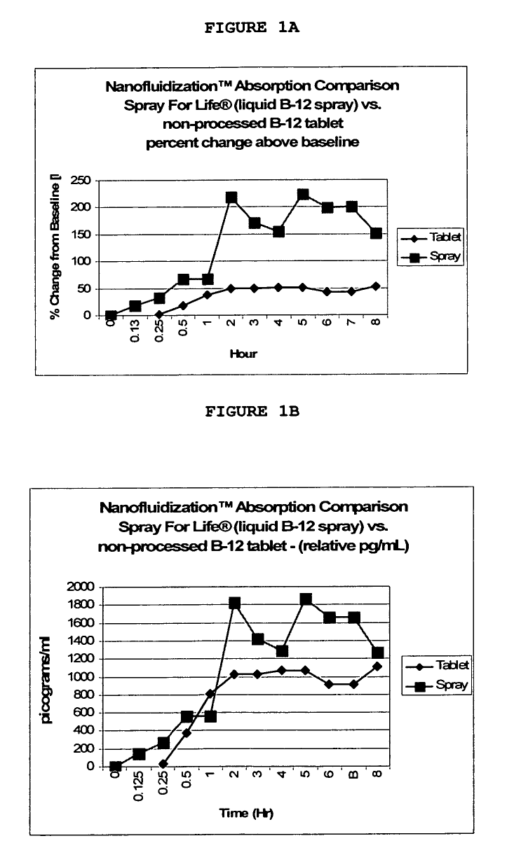

FIG. 1A is a graphic comparison of mucosal absorption of nanofluidized B-12 versus gastro-intestinal absorption via oral administration of a well recognized commercially available B-12 in tablet form, and is indicative of the relative and respective increase in circulating B-12 concentration in picograms/mL as a function of time, from an initial baseline.

FIG. 1B represents a graphic analysis of the data from the test subject, illustrating the plasma concentration curves for the nanoprocessed vitamin B-12 absorbed via the buccal mucosa as compared to vitamin B-12 in tablet form absorbed via the gastro-intestinal tract. Based on this preliminary clinical trial, the results demonstrate that nanoprocessed vitamin B-12 was absorbed in significantly higher amounts than the well recognized commercially available vitamin B-12 tablet and had a faster onset of action than the vitamin B-12 tablet. In addition, The Area-Under-the-Curve (AUC) for nanoprocessed vitamin B-12, in FIG. 1 shows over 47% greater absorption in the nanoprocessed vitamin B-12 than that of the vitamin B-12 tablet.

Thus, it is seen that administration of vitamin B-12 in a nanofluidized suspension absorbed via the buccal mucosa results in substantially higher absorption at a substantially higher rate than that absorbed via gastrointestinal absorption without engendering swallowing difficulties and digestibility issues, which has heretofore been lacking in the prior art.

### Example 2

Study of the effects of nanoprocessed vitamin B-12 in human patients suffering from vitamin B-12 deficiency or pernicious anemia.

Objective:

To establish the effectiveness of the nanoprocessed SPRAY FOR LIFE vitamin B-12 Energy Booster of the instant invention in reducing or eliminating erythrocyte i.e., red blood cell, (RBC) abnormalities of size and shape in patients with pernicious anemia induced by vitamin B-12 deficiency. Samples of the nanoprocessed SPRAY FOR LIFE vitamin B-12 Energy Booster of the instant invention were produced and administered to the sublingual mucosal membranes of three different human patients by a spray applicator.

Methods:

The three human subjects used in this study were tested twice. The test subjects used had not taken any other supplements containing B-12 for one month prior to initial testing or between the initial and final test visit.

The initial test was used to establish any blood cell abnormalities in each patient. The second test was conducted after all the patients had used a spray applicator to administer approximately 3 sprays of nanoprocessed SPRAY FOR LIFE vitamin B-12 Energy Booster of the present invention by carefully spraying sublingually (under the tongue), two times per day for 30 days, for a total dose of approximately 1200 mcg of vitamin B-12 per day.

During each test visit, each patient had approximately 5 ml of blood (SST tubes) drawn by routine venipuncture to establish a baseline (pre-dosing and post-dosing).

Next, the patients' whole blood samples where shipped, on ice, to the same analytical laboratory (LabOne, Inc) for blood cell morphology testing.

Red blood cell manual morphology technique was used to determine any red blood cell abnormalities that may be present in the patients' blood samples. In this technique the size and shape of the red blood cells are measured by machine and counted manually under a high-powered microscope, such as Leica Microstar IV microscope, by a trained and skilled technician

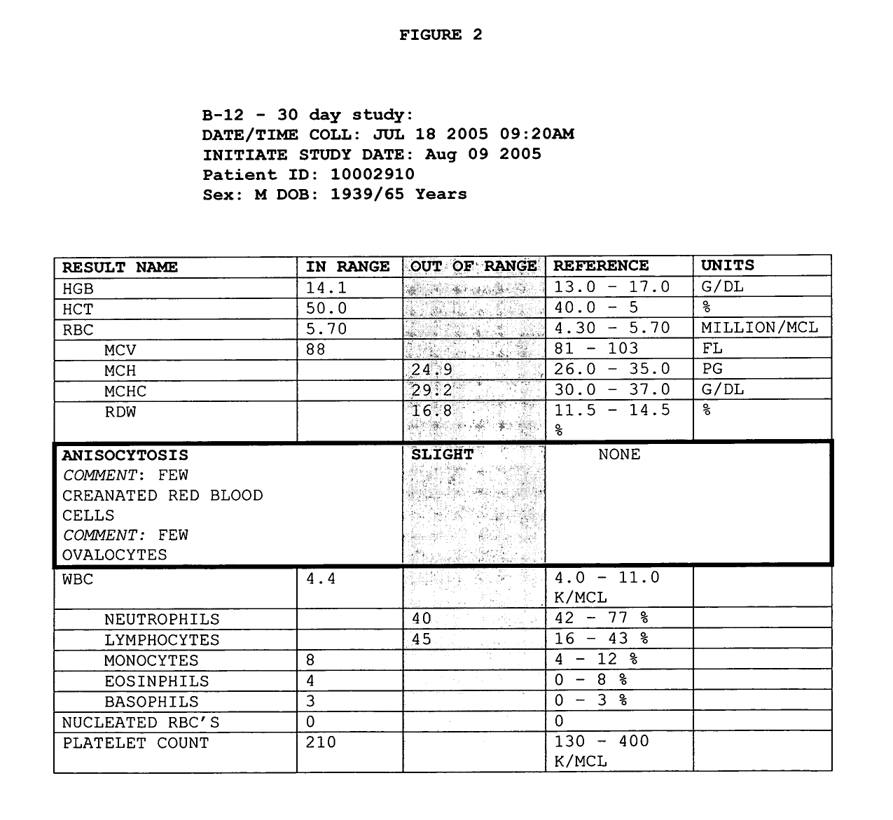

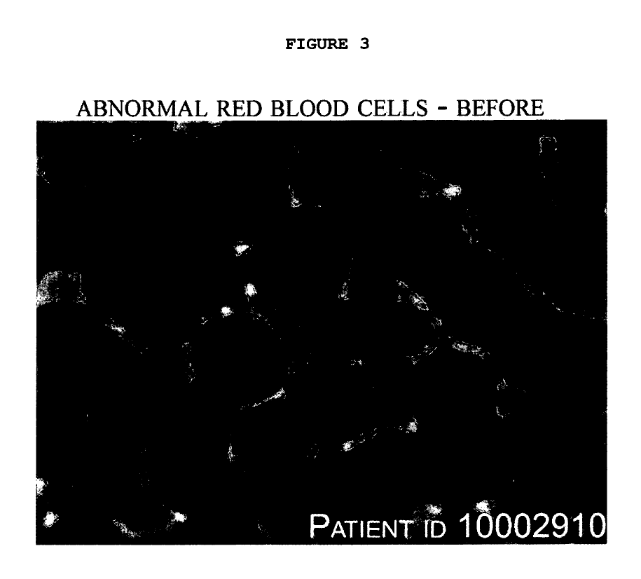

The first patient tested in this study (patient ID NO: 10002910), a 65-year-old male, diagnosed with pernicious anemia complained of being tired and depressed. An initial test on the patient's whole blood sample was conducted and analyzed by red blood cell manual morphology technique. The initial results of the red blood cell manual morphology technique revealed two abnormalities, slight anisocytosis (red blood cell size is too small as compared to normal size range) and a few ovalocytes (oval shaped red blood cells rather than round), as illustrated in the summary of the hematology report (FIG. 2) and the photograph of the sample test slide (FIG. 3) using a high-powered microscope.

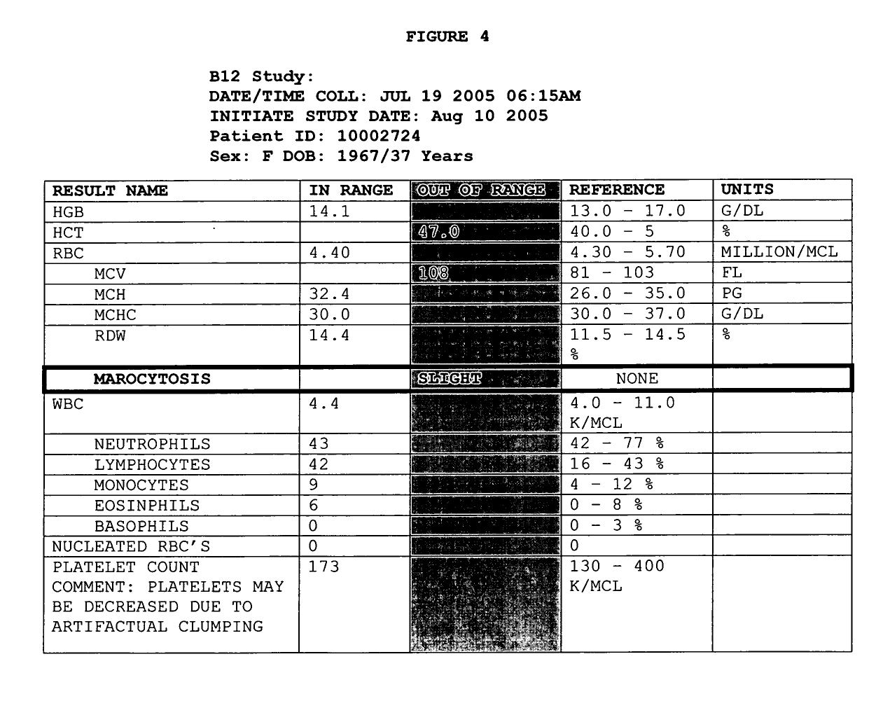

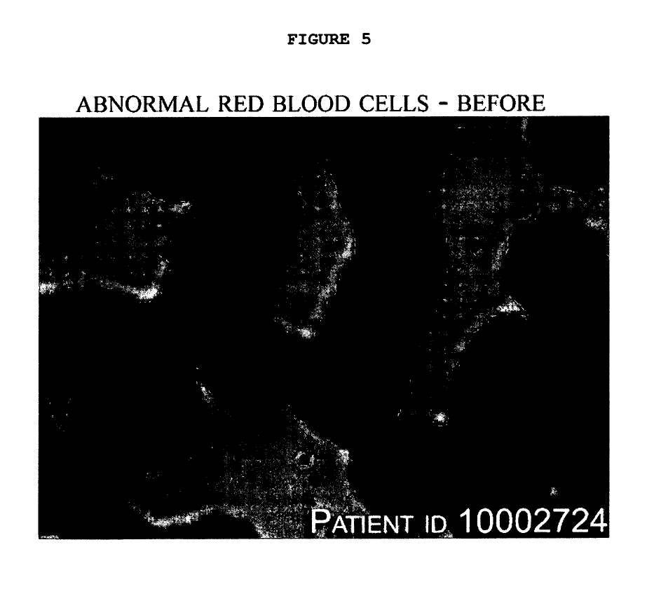

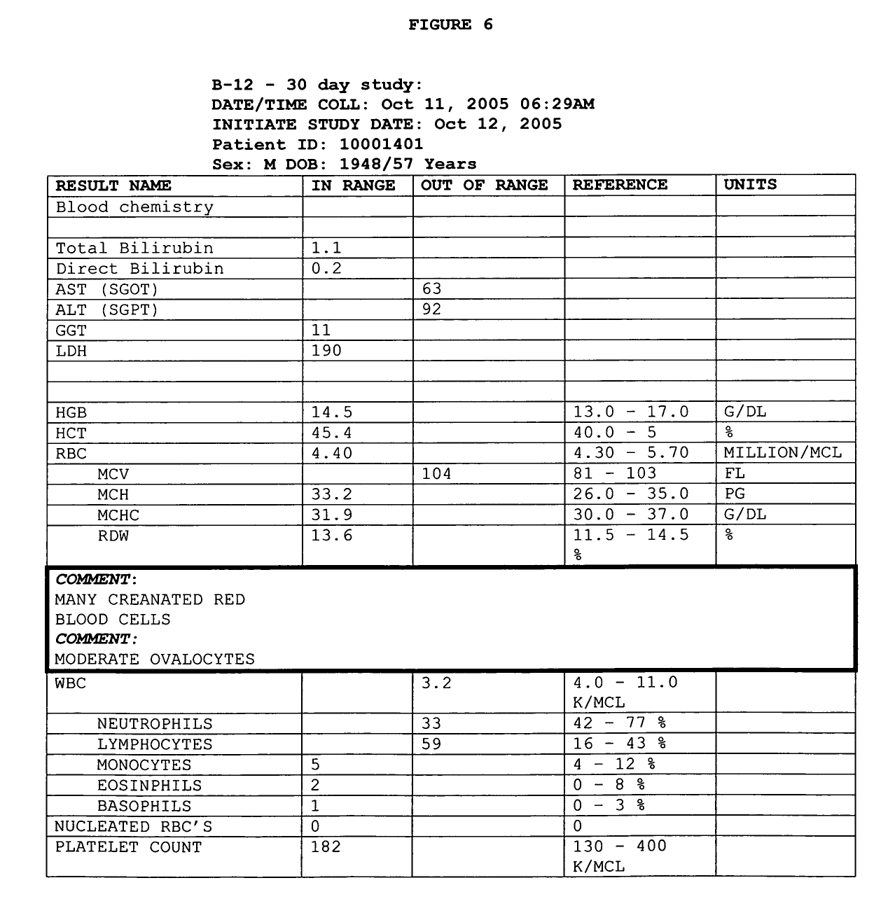

The second patient used in this study (patient ID NO: 10002724), a 37-year-old female, is an avid athlete diagnosed with pernicious anemia induced by B-12 deficiency. The second patient suffered with “restless leg” symptoms, less than optimum recovery time after workouts, and muscle cramps. As with the first patient, the initial test was conducted and analyzed by way of red blood cell manual morphology technique the following day. The test results of the red blood cell manual morphology technique discovered slight macrocytosis, that is, the red blood cells are too fat or large resulting in poor delivery of oxygen to other cells, as illustrated in the summary of the hematology report (FIG.4) and the photograph of the sample test slide (FIG. 5) using a high powered microscope.

The third patient used in this study (patient ID NO: 10001401), a 57-year-old male, diagnosed with a Vitamin B-12 deficiency complained of having a lack of energy. An initial test on the patient's whole blood sample demonstrated altered liver functions with an AST (SGOT) value of 63 and an ALT (SGPT) value of 92, both of which are out of normal range and indicate potential liver damage. In addition the patient's mean cell volume (MCV) was out of range at 104. The patient's hemoglobin at the time was 14.5 and the hematocrit was 45.4.

The second tests were conducted on the three patients after having sublingually administered the sprays for the 30 day treatment period and substantially the same testing protocol was followed for all the patients as was performed during the first visit. During the aforementioned treatment period, no change was made to either of the patient's eating plan, exercise program, or supplement program except for the introduction of the instant vitamin B-12 spray.

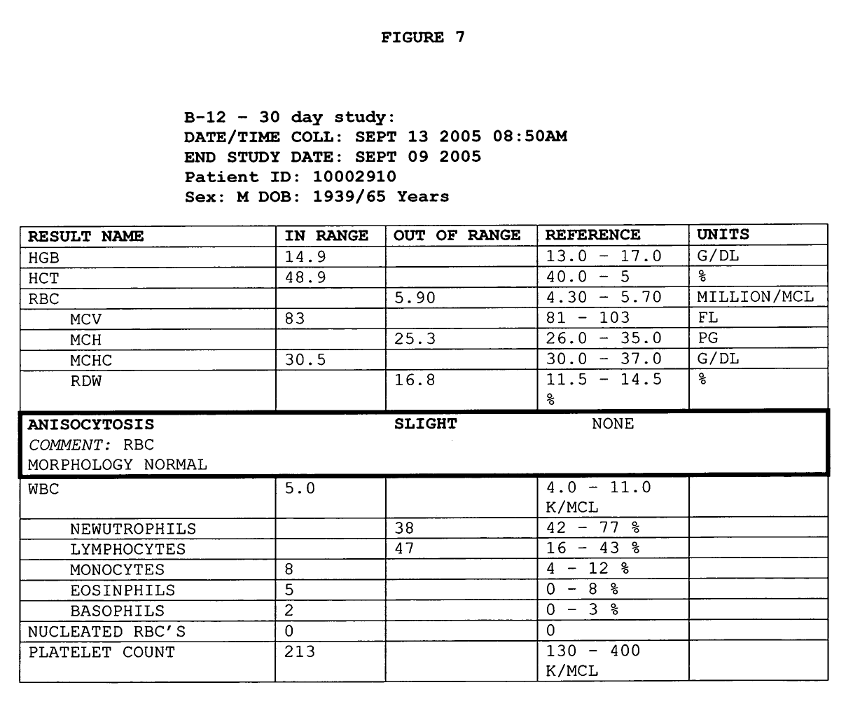

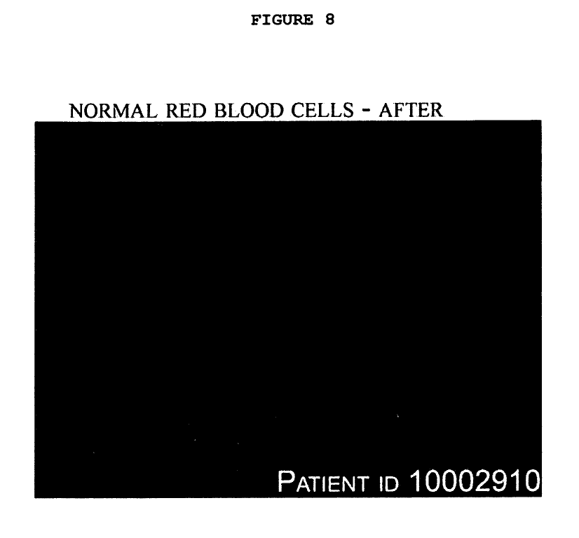

For the first patient (65-year-old male), the second test results on the whole blood sample revealed that not only had the size of all the tested red blood cells fallen within the normal range and shape, but the number of red blood cells and hemoglobin level had noticeably improved, as illustrated in the summary of the hematology report (FIG. 7) and the photograph of the sample test slide (FIG. 8) taken during the second red blood cell manual morphology procedure using a high-powered microscope. In fact, the patient had commented that he experienced a higher energy level and little or no depression within the first week of treatment.

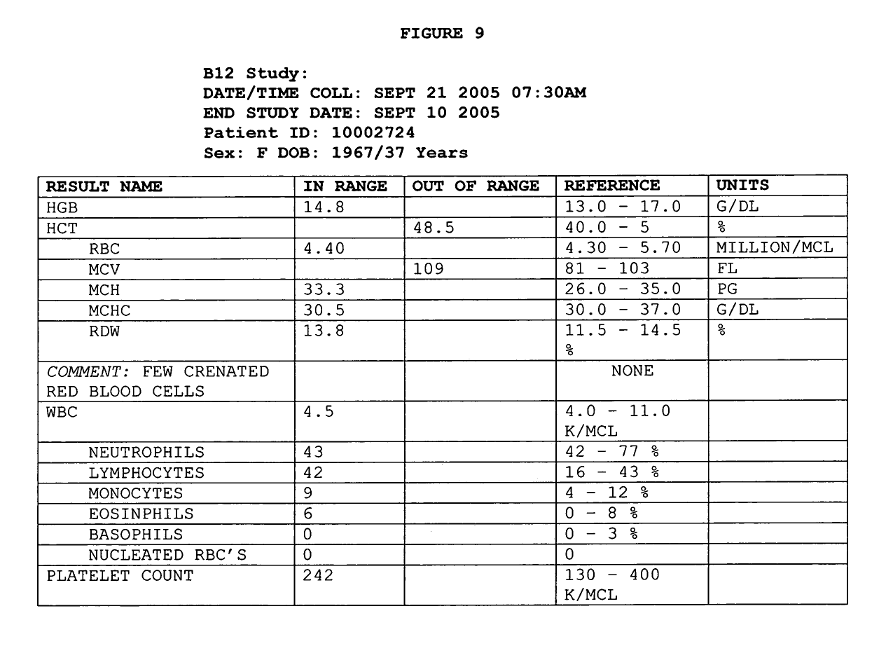

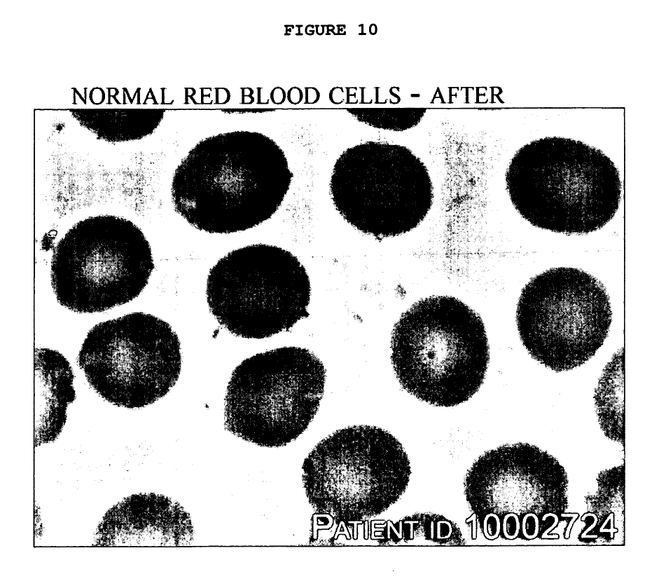

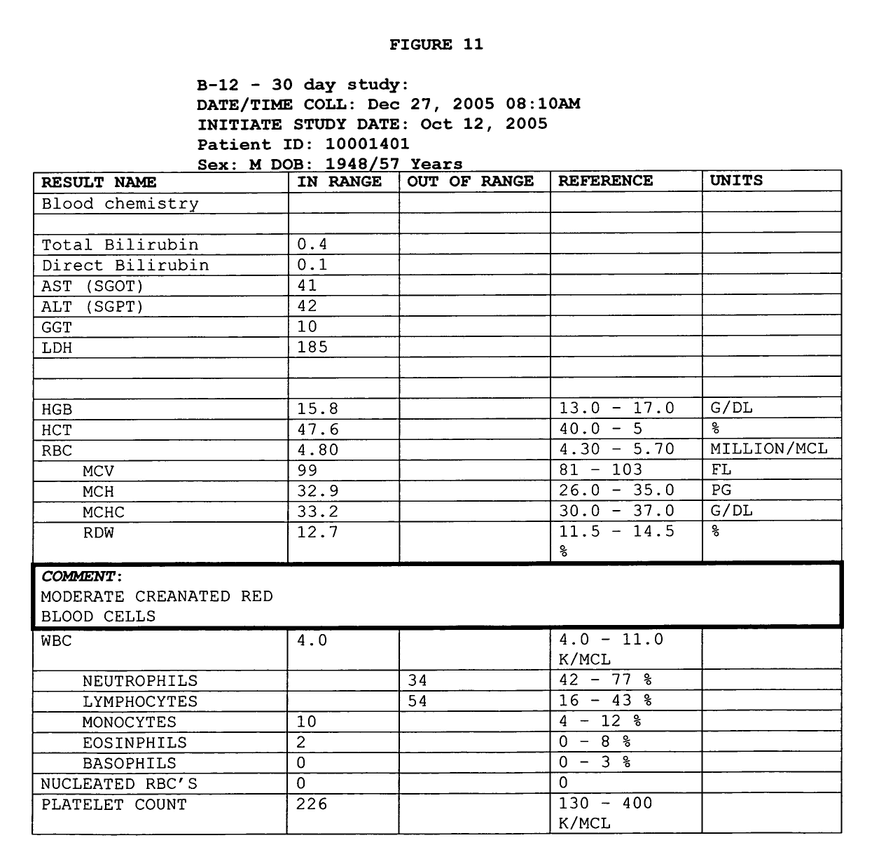

The second patient (37-year-old female) maintained her extensive exercise program throughout the 30-day testing period; during which, she noticed marked improvement in her recovery times. Also, she noticed less muscle cramping and irritation. The results from the second patient's hematology report on the whole blood sample (FIG. 9) and the photograph of the sample test slide taken during the second red blood cell manual morphology procedure (FIG. 10) illustrate a return to normal red blood cell size and no macrocytosis was found. Although no improvement in the number of red blood cells were observed in the second patient, there was a marked improvement in hemoglobin levels.

For the third patient (57-year-old male), another blood sample was taken approximately 6 weeks after the 30 day treatment with vitamin B12 lingual spray was initiated. Testing of this sample showed that the liver functions had returned to normal, with AST level at 41 and the ALT level at 42, both within the normal range of liver functions. In addition the hemoglobin level had improved from 14.5 to 15.8 and the hematocrit had risen from 45.4 to 47.6, both of which were the highest levels seen in this patient in over two years. The mean cell volume also returned to a normal level.

These findings indicate that this patient's red blood cell functions and liver functions had been markedly improved by the course of vitamin B12 therapy, both returning to normal levels which had not been seen throughout the course of his disease. In addition, the patient experienced an increase in energy levels, “felt better,” and had improved overall health.

Normally, red blood cell morphology responds to sublingual tablet, or injection, of vitamin B-12 supplements in 90 to 120 days, not within the accelerated time frame of about 30 days, as evidenced by the instant experiments. While not wishing to be bound to any particular theory, it is reasonable for the skilled artisan to conclude from the results of the three examples set forth above that the nanodispersions of the present invention allow molecules to be delivered across transmucosal tissue (i.e. sublingual) barriers at an increased rate and with reduced degradation than conventional non-processed solutions. This, in turn, preserves the potency and therapeutic effects of B-12 in maintaining proper biological processes, for example, red blood cell maturation, development and normalization of function, (e.g., hemoglobin synthesis and oxygen transport) as seen above.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A method for administration of a nanofluidized nanosuspension containing vitamin B-12 to a subject via a transmucosal route comprising:
forming, via a nanofluidization process, a stable nanosuspension comprising nanodroplets of said vitamin B-12; and
contacting said nanosuspension with the oral mucosal membranes of said subject;
wherein said nanosuspension containing vitamin B-12 is absorbed into the bloodstream of said subject.

2. The method of claim 1, wherein said subject is a human.

3. The method of claim 1, wherein said subject is an animal.

4. The method of claim 1, wherein said nanosuspension contains particles in a size range of about 87 nm to about 10 μm.

5. The method of claim 1, wherein said nanosuspension further includes a permeation enhancer.

6. The method of claim 1, wherein said nanosuspension is in the form of a nanofluidized spray, an aerosol, a tablet, a capsule, a pill, a liquid, a suppository, or a gel.

7. A method for administration of a nanofluidized suspension containing vitamin B-12 to a subject comprising:
forming, via a nanofluidization process, a stable nanosuspension comprising nanodroplets of said vitamin B-12; and
administering said nanosuspension to said subject;
wherein said nanosuspension containing vitamin B-12 is absorbed into the bloodstream of said subject.

8. The method of claim 7, wherein said subject is a human.

9. The method of claim 7, wherein said subject is an animal.

10. The method of claim 7, wherein said nanosuspension contains particles in a size range of about 87 nm to about 10 μm.

11. The method of claim 7, wherein said nanosuspension further includes a permeation enhancer.

12. The method of claim 7, wherein said nanosuspension containing vitamin B-12 additionally contains at least one additional biologically active agent.

13. The method of claim 7, wherein said biologically active agent is at least one pharmaceutical agent, at least one nutritional product, or a combination thereof.

14. The method of claim 7, wherein said method of administration is at least one method selected from the group consisting of parenteral, intrathecal, intravenous, transdermal, transmucosal, or combinations thereof.

15. The method of claim 7, wherein said nanosuspension is in the form of a nanofluidized spray, an aerosol, a tablet, a capsule, a pill, a liquid, a suppository, or a gel.

16. A method for ameliorating symptoms of pernicious anemia in a subject displaying said symptoms comprising:
providing a nanosuspension containing vitamin B-12 in a size range of about 87 nm to about 10 μm; and
contacting said nanosuspension with the oral mucosal membranes of said subject;
wherein said nanosuspension containing vitamin B-12 is absorbed into the bloodstream of said subject.

17. The method of claim 16, wherein said nanosuspension containing vitamin B-12 additionally contains at least one additional biologically active agent.

18. The method of claim 17, wherein said biologically active agent is at least one pharmaceutical agent, at least one nutritional product, or a combination thereof.

19. The method of claim 16, wherein said nanosuspension further includes a permeation enhancer.

20. An in vivo process for accelerated formation, maturation, and normalization of red blood cells comprising:
providing a nanosuspension containing vitamin B-12 in a size range of about 87 nm to about 10 μm; and
contacting said nanosuspension with the oral mucosal membranes of a subject, whereby said nanosuspension containing vitamin B-12 is absorbed into the bloodstream of said subject;
wherein mature red blood cells of normal size and shaped are formulated within an accelerated time period.

21. A composition, useful in the treatment of vitamin B-12 deficiencies (including pernicious anemia), comprising a nanosuspension of vitamin B-12, wherein said vitamin B-12 is present in a size range of about 87 nm to about 10 μm.

22. The composition of claim 21, further including at least one additional biologically active agent.

23. The composition of claim 21, wherein said biologically active agent is at least one pharmaceutical agent, at least one nutritional product, or a combination thereof.

24. The composition of claim 21, wherein said nanosuspension further includes a permeation enhancer.
